(19) United States
(12) Patent Application Publication
(10) Pub. No.: US 2018/0116714 A1
(43) Pub. Date: May 3, 2018

(54) MEDICAL DEVICE WITH A SHAPE-MEMORY ALLOY COATED INNER SURFACE

(71) Applicant: Cook Medical Technologies LLC, Bloomington, IN (US)

(72) Inventor: John Karpiel, Winston-Salem, NC (US)

(73) Assignee: Cook Medical Technologies LLC, Bloomington, IN (US)

(21) Appl. No.: 15/795,838

(22) Filed: Oct. 27, 2017

Related U.S. Application Data
(60) Provisional application No. 62/415,208, filed on Oct. 31, 2016.

(51) Int. Cl. A61B 18/14

(52) U.S. Cl. CPC A61B 18/1492; A61B 18/1206; A61B 2018/144; A61B 2018/1465

(57) ABSTRACT

A medical device may include an elongate tubular member having a distal portion that is configured to curl in a desired direction. A shape-memory alloy structure may be disposed in a lumen of the elongate tubular member at the distal portion. The shape-memory alloy may have an original, pre-deformed shape that is curved to facilitate the curling of the distal portion in the desired direction.

### CROSS-REFERENCE TO RELATED APPLICATIONS

This application claims the benefit of U.S. Provisional Application No. 62/415,208, filed Oct. 31, 2016. The contents of U.S. Provisional Application No. 62/415,208 are incorporated by reference in their entirety.

### TECHNICAL FIELD

The present invention relates generally to medical devices, and more particularly to sphincterotomes.

### BACKGROUND

A sphincterotome is a medical device that is used to perform a sphincterotomy, which involves cutting a sphincter muscle, such as the sphincter of Oddi. The sphincter muscle may need to be cut to relieve its constrictive nature and allow one or more medical devices through the muscle. For example, problems occurring in the biliary tree, such as the formation of bile duct stones or papillary stenosis, may be treated using medical devices that are delivered into the biliary tree. In order to access the biliary tree, the medical devices may pass through the sphincter of Oddi. To facilitate passage of the medical devices through the sphincter of Oddi, the sphincter muscle may be cut using a sphincterotome.

A sphincterotome may generally include an elongate tubular member, such as a catheter, and a cutting wire that is used to cut the sphincter muscle. The cutting wire may extend through a lumen of the catheter, except at a distal portion of the catheter, where the cutting wire may project from and be exposed outside of the catheter. The exposed portion, which may be referred to as a cutting edge, may be used to cut the sphincter muscle.

A sphincterotomy generally involves a two-part process: cannulation of the biliary tree and cutting the sphincter muscle by sending electric current through the cutting wire. Cannulation of the biliary tree may include inserting the distal portion of the catheter into the papilla and using the distal portion and the cutting edge to lift an upper portion (i.e., the roof) of the papilla. The roof of the papilla may be lifted by proximally pulling the cutting wire taut, causing the distal portion of the tubular member to bow or curl to form an arc. After cannulation, the electric current may be provided to the cutting edge to cut the sphincter muscle.

When performing the sphincterotomy, the sphincter muscle is often viewed relative to a clock face, and the roof of the sphincter is typically positioned where the number “12” is located. It is then often desirable to lift and cut the roof of the papilla with the cutting edge in the “12 o'clock” position, i.e., with the distal portion having curled toward and the cutting edge being aligned with the “12” of the clock face, or at least within a range, such as between the “11” and the “1” of the clock face. However, it is often difficult to curl the distal portion in the 12 o'clock direction for sphincterotomes that rely on the proximal pulling of the cutting wire to also curl the distal portion. As such, ways to encourage or facilitate the curling of the distal portion in a desired radial direction (e.g., a 12 o'clock direction) are desirable.

### BRIEF SUMMARY

The present description describes medical devices that include an elongate shape-memory alloy (SMA) component or structure disposed on an inner surface of an elongate tubular member. In one embodiment, a medical device includes an elongate tubular member and an elongate shape-memory alloy (SMA) structure. The elongate tubular member longitudinally extends from a proximal portion to a distal portion, and includes a body and a lumen longitudinally extending in the body from the proximal portion to the distal portion, where an inner surface of the body defines the lumen. The elongate shape-memory alloy structure is disposed on the inner surface at the distal portion and has a longitudinally-curved pre-deformed shape. The distal portion of the elongate tubular member is configured to be in a curled state due to the longitudinally-curved pre-deformed shape of the elongate SMA structure.

In another embodiment, a medical device includes an elongate tubular member that longitudinally extends from a proximal portion to a distal portion. The elongate tubular member includes: a body; a cutting wire lumen and an additional lumen, each longitudinally extending in the body from the proximal portion to the distal portion; a cutting wire longitudinally extending in the cutting wire lumen, wherein a cutting edge of the cutting wire longitudinally extends outside of the body; and an elongate shape-memory alloy (SMA) structure disposed on an inner surface of the body that defines the additional lumen. The elongate SMA structure has a longitudinally-curved pre-deformed shape, and the distal portion of the elongate tubular member is configured to be in a curled position due to the longitudinally-curved pre-deformed shape of the elongate SMA structure.

In some embodiments, the elongate SMA structure includes a curved cross-sectional profile.

In some embodiments, a contour of the curved cross-sectional profile is defined by the inner surface of the body.

In some embodiments, the curved cross-sectional profile is an arc.

In some embodiments, the curved cross-sectional profile is a circle.

In some embodiments, the elongate SMA structure includes an elongate tubular structure comprising a body and a lumen longitudinally extending in the body.

In some embodiments, the elongate tubular structure includes a series of linearly-aligned notches.

In some embodiments, each of the linearly-aligned notches has a circumferential length less than or equal to half a circumferential length of the elongate tubular structure.

In some embodiments, a second lumen longitudinally extends in the body, and a cutting wire is movably disposed and extending in the body.

In some embodiments, a cutting edge of the cutting wire longitudinally extends to outside of the body at the distal portion, and the elongate SMA structure longitudinally extends alongside the cutting edge.

In some embodiments, the elongate SMA structure includes nitinol.

In some embodiments, the additional lumen includes a wireguide lumen.

In some embodiments, the additional lumen includes a contrast lumen.

In some embodiments, the additional lumen includes a first additional lumen and the elongate tubular member includes a second additional lumen.

Other embodiments are possible, and each of the embodiments can be used alone or together in combination. Accordingly, various embodiments will now be described with reference to the attached drawings.

### BRIEF DESCRIPTION OF THE DRAWINGS

FIG. 1A shows a cross-sectional side view of a sphincterotome connected to a power source, with a cutting edge in a relaxed position.

FIG. 1B shows a cross-sectional side view of the sphincterotome of FIG. 1A, with the cutting edge in a cutting position.

FIG. 2 shows a distal portion of the sphincterotome of FIGS. 1A and 1B at a treatment site within a patient.

FIG. 3 shows a cross-sectional axial view of an example cross-sectional profile of the sphincterotome of FIGS. 1A and 1B, with an elongate smart-memory alloy structure having a circular cross-section disposed in a wireguide lumen.

FIG. 4 shows a perspective view of a first example configuration of an elongate shape-memory alloy structure implemented in the sphincterotome of FIGS. 1A and 1B.

FIG. 5 shows a perspective view of a second example configuration of the elongate shape-memory alloy structure implemented in the sphincterotome of FIGS. 1A and 1B.

FIG. 6 shows a perspective view of a third example configuration of the elongate shape-memory alloy structure implemented in the sphincterotome of FIGS. 1A and 1B.

FIG. 7 shows a perspective view of a fourth example configuration of an elongate shape-memory alloy structure implemented in the sphincterotome of FIGS. 1A and 1B.

FIG. 8 shows a cross-sectional axial view of another example cross-sectional profile of the sphincterotome of FIGS. 1A and 1B, with an elongate smart-memory alloy structure having an arc-shaped cross-section disposed in a wireguide lumen.

FIG. 9 shows a cross-sectional axial view of another example cross-sectional profile of the sphincterotome of FIGS. 1A and 1B, with an elongate smart-memory alloy structure having a circular cross-section disposed in a contrast lumen.

FIG. 10 shows a cross-sectional axial view of another example cross-sectional profile of the sphincterotome of FIGS. 1A and 1B, with an elongate smart-memory alloy structure having an arc-shaped cross-section disposed in a contrast lumen.

### DETAILED DESCRIPTION

The present description describes medical devices that include an elongate shape-memory alloy (SMA) component or structure disposed on an inner surface of an elongate tubular member. The SMA structure is configured to facilitate the curling of a distal portion of the elongate tubular member in a desired direction. The present description describes the elongate SMA structure as being implemented with a sphincterotome. However, devices other than sphincterotomes, including those having a distal portion that curls in a desired direction, may similarly by implemented with the described elongate SMA structure.

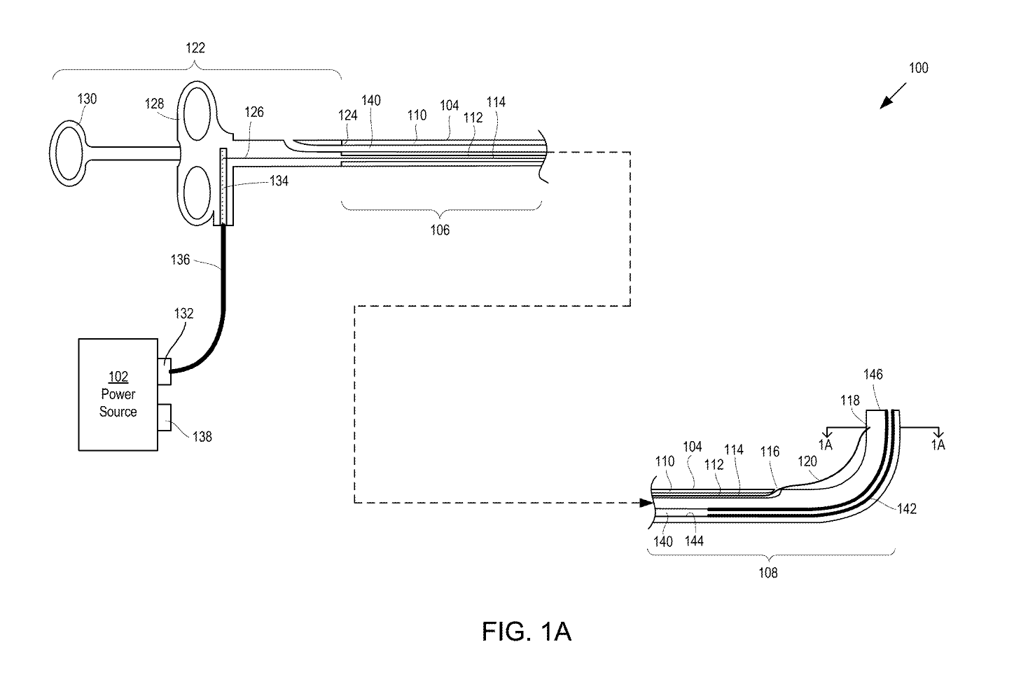

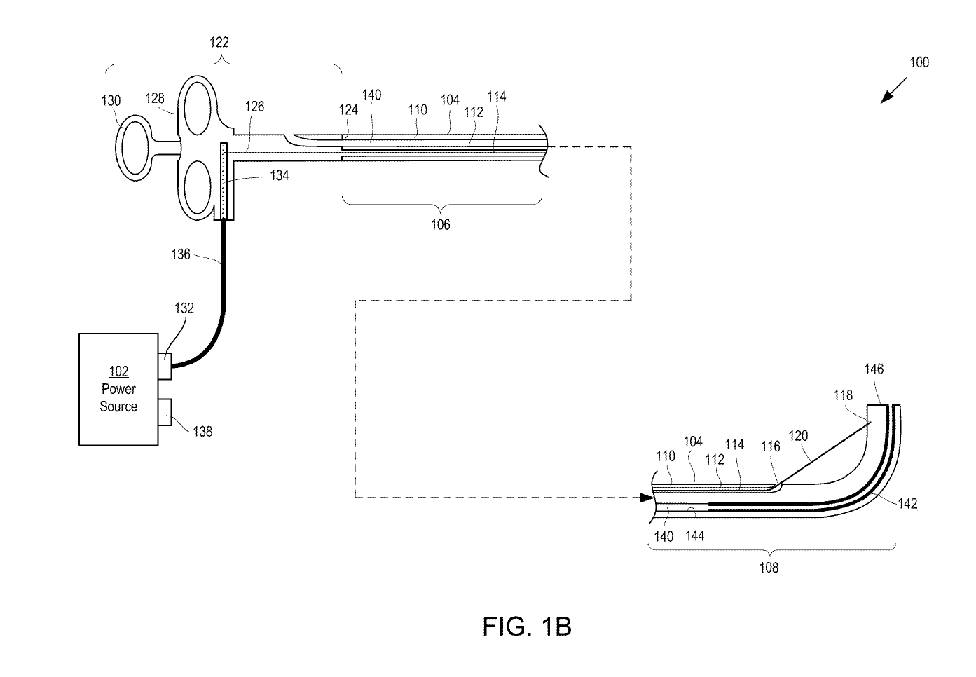

FIGS. 1A and 1B show cross-sectional side views of a sphincterotome 100 electrically connected to a power source 102. The sphincterotome 100 may include an elongate tubular member 104, such as a catheter, that extends from a proximal portion 106 to a distal portion 108. The elongate tubular member 104 may include a body 110 and a cutting wire lumen 112 longitudinally extending in the body 110 from the proximal portion 106 to the distal portion 108. A cutting or active wire 114 used to cut a sphincter muscle at a treatment site within a patient may be disposed within the cutting wire lumen 112. The cutting wire 114 may longitudinally extend in the cutting wire lumen 112 from the proximal portion 106 to the distal portion 108. At the distal portion 108, the cutting wire 114 may extend or protrude from the cutting wire lumen 112 inside the body 110, through a first opening 116 of the tubular member 104, to outside the tubular member 104. Outside the tubular member 104, the cutting wire 114 may longitudinally extend substantially parallel with the tubular member 104 to a second opening or anchor point 118 that is distal the first opening 116, where a distal end of the cutting wire 114 may re-enter and/or be fixedly attached to the tubular member 104. The portion of the cutting wire 114 outside of the tubular member 104 at the distal portion 108 may be referred to as a cutting edge 120, which may be the portion of the cutting wire 114 that cuts the sphincter muscle.

The cutting edge 120 may move between a cutting position and a relaxed position. The cutting edge 120 may be positioned in the cutting position when a user of the sphincterotome 100 intends to lift the roof of the papilla and/or cut the sphincter muscle. The cutting edge 120 may be positioned in the relaxed position when the user intends to perform an action other than lifting the roof of the papilla or cut the sphincter muscle, such as delivering the distal portion 108 to and from the treatment site or cannulating the biliary tree, as examples. FIG. 1A shows the cutting edge 120 in the relaxed position. In the relaxed position, the cutting edge 120 may have relatively little tension. The cutting edge 120 may be moved from the relaxed position to the cutting position by proximally pulling the cutting wire 114 taut. FIG. 1B shows the cutting wire 114 taut and the cutting edge 120 in the cutting position.

The sphincterotome 100 may further include a handle assembly 122 connected to a proximal end 124 of the elongate tubular member 104. A proximal end 126 of the cutting wire 114 may be operably connected to the handle assembly 122. The handle assembly 122 may be configured to move the cutting edge 120 between the relaxed and cutting positions. In the example configuration shown in FIGS. 1A and 1B, the handle assembly 122 may include a gripping assembly that includes a first gripping portion 128 for an operator's fingers and a second gripping portion 130 for the operator's thumb. The operator may configure the cutting edge 120 in the relaxed position by moving the first gripping portion 128 and the second gripping portion 130 away from each other (FIG. 1A), and may configure the cutting edge 130 in the cutting position (i.e., may pull the cutting wire 114 taut) by moving the first gripping portion 128 and the second gripping portion 130 close together (FIG. 1B). This handle configuration is merely exemplary and other handle assembly configurations for a sphincterotome 100 may be possible.

The cutting wire 114 may be part of an active electrical path that is configured to conduct and deliver electrical current to the cutting edge 120 to cut the sphincter muscle at the treatment site. To conduct and deliver the current, the cutting wire 114 may be electrically coupled to the power source 102. The power source 102 may be configured to generate and/or supply electrical current to the cutting wire 114. Examples of the power source 104 may be a radio frequency (RF) generator or an electrosurgical unit (ESU). For some example configurations, as shown in FIGS. 1A and 1B, the handle assembly 122 may be used to electrically couple the active path to an active port 132 of the power source 102. For example, the proximal end 126 of the cutting wire 114 may be connected to a conductive member 134 of the handle assembly 122, which in turn, may be configured to be electrically connected to electrical cabling 136 that is configured to electrically and physically connect to the handle assembly 122 and the active port 132 of the power source 102. Ways of electrically connecting the cutting wire 114 to the power source 102 other than through the handle assembly 122 may be possible.

In addition, the electrical current that is supplied may be returned back to the power source 104 using a return path (not shown in FIGS. 1A and 1B). For some example configurations, the sphincterotome 100 may have a monopolar configuration, in which the return path may include a neutral electrode (not shown) positioned on the patient and electrically coupled to a return port 138 of the power source 102. For other example configurations, the sphincterotome 100 may have a bipolar configuration, in which the return path may longitudinally extend within and/or alongside the elongate tubular member 104 back to the return port 132.

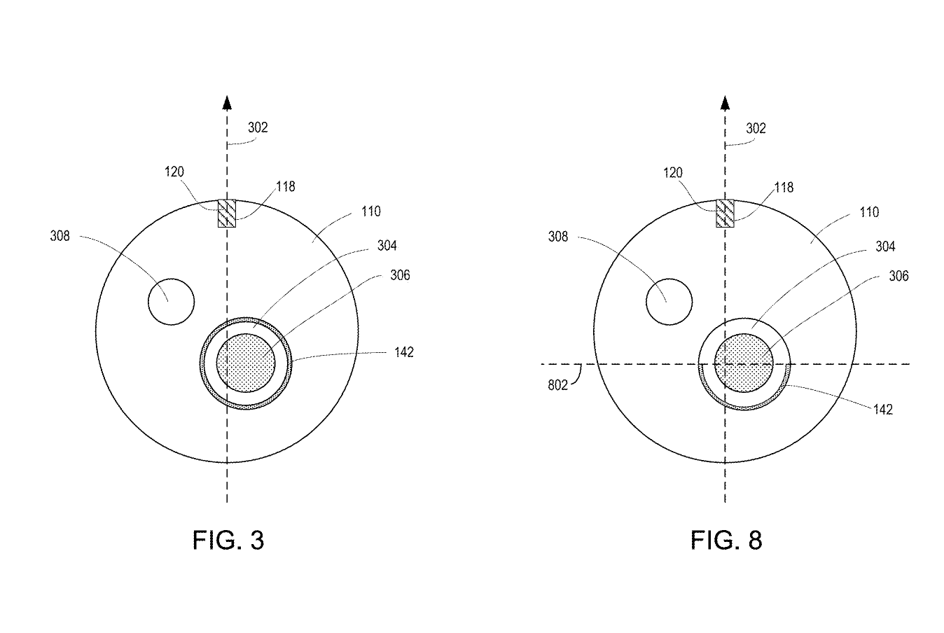

The elongate tubular member 104 may further include at least one additional lumen 140, in addition to the cutting wire lumen 112, that longitudinally extends in the body 110 from the proximal portion 106 to the distal portion 108. For some example configurations, the additional lumen 140 may be a wireguide lumen configured to receive and have movably disposed therein a wireguide (not shown in FIGS. 1A and 1B) that facilitates delivery of the sphincterotome 100 to and from the treatment site. For delivery of the distal portion 108 to the treatment site within the patient, a distal end of an endoscope may be delivered to the treatment site within the patient. A wireguide may then be inserted into a working channel of the endoscope and distally advanced until it is at the treatment site. Additionally, a distal end of the wireguide may be positioned into and past the sphincter muscle to be cut. The sphincterotome 100 may then be inserted into the working channel by positioning the wireguide lumen 140 over or about the wireguide and distally advancing the elongate tubular member 104 until the distal portion 108 exits the working channel and reaches the treatment site. The wireguide may then be proximally retracted so that the distal end of the wireguide is away from the treatment site and the cutting portion of the sphincterotomy is then performed. For other example configurations, the additional lumen 140 may be a contrast lumen configured to deliver contrast to the treatment site. For still other example configurations, the elongate tubular member 104 may include both a wireguide lumen and a contrast lumen, as shown and described in further detail with reference to FIGS. 3 and 8-10.

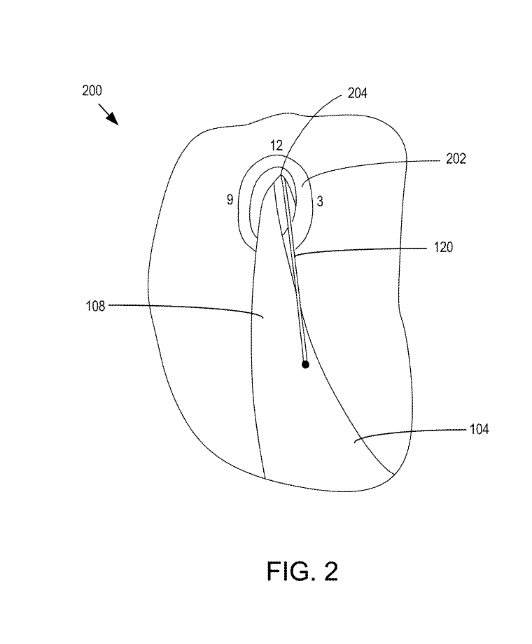

FIG. 2 shows the distal portion 108 of the elongate tubular member 104 delivered to a treatment site 200, where the cutting edge 120 is to cut a sphincter muscle 202. When performing a sphincterotomy, the sphincter muscle 202 is often oriented with reference to a clock face. FIG. 2 shows the 12 o'clock, 3 o'clock, and 9 o'clock positions of the clock face. To cut the sphincter muscle 202, it may be desirable to lift and cut a portion 204 of the sphincter muscle 202 and or adjacent tissue 204 (e.g., the duodenal papilla) located at or near the 12 o'clock position. To do so, it may be desirable to align the cutting edge 120 in the 12 o'clock position and curl the distal portion 108 in the 12 o'clock direction, as shown in FIG. 2.

As previously described, the cutting wire 114 may be anchored or fixedly attached to the body 110 of the elongate tubular member 104 at an anchor point 118. As such, operating the handle assembly 122 to position in the cutting edge 120 in the taut, cutting position may also proximally pull on a distal end 146 of the body 110, causing the distal portion 108 to curl. However, due to torquing, it is often difficult to have the distal portion 108 curl in the desired 12 o'clock direction for configurations that rely solely on the cutting wire 114 to perform the curling. Instead, the distal portion 108 may tend to veer off to the side towards the 3 o'clock or 9 o'clock positions.

Referring back to FIGS. 1A and 1B, the sphincterotome 100 may include an elongate smart-memory alloy (SMA) structure or component 142 disposed on, affixed to, and/or coating an inner surface 144 of the body 110 that defines the additional lumen 140. As shown in FIGS. 1A and 1B, the elongate SMA structure 142 maybe disposed at the distal portion 108. An example smart-memory alloy material may be nitinol, although other materials may be possible.

For some example configurations, the elongate SMA structure 142 may be sized to be force fit into the additional lumen 140 so that the elongate SMA structure 142 is fixedly attached to the body 110 while disposed in the additional lumen 140, although other ways of affixing the elongate SMA structure 142 to the inner surface 144 may be possible. Also, a cross-sectional profile of the elongate SMA structure 142 may conform to a shape of a boundary of the additional lumen 140 as defined by the inner surface 144. For example, if the additional lumen 140 has a circular cross-sectional profile, then the cross-sectional profile of the elongate SMA structure 142 may have a curved profile conforming to the circular cross-sectional profile of the additional lumen 140.

In general, the cross-sectional profile of the elongate SMA structure 142 may be relatively thin in relation to the additional lumen 140 in which the elongate SMA structure 142 is disposed. For example, the cross-sectional area of the elongate SMA structure 142 may be less than the cross-sectional area of the additional lumen 140. As such, the elongate SMA structure 142 may occupy less than the entire space or volume of the additional lumen 140. By having a cross-sectional shape that occupies less than the entire cross-sectional area of the additional lumen 140 and that conforms to the shape of the boundary of the additional lumen 140, the additional lumen 140 may still be used according to its intended function. For example, where the additional lumen 140 is a wireguide lumen, the additional lumen 140 may still be operable to receive and have moveably disposed therein a wireguide despite also having disposed therein the elongate SMA structure 142. This is shown and described in further detail with respect to FIGS. 3 and 8-10.

The embodiments of the present description may be in contrast to other embodiments that utilize an elongate structure to encourage bending in a desired direction which have a cross-sectional shape and size that, if disposed in the additional lumen 140, would prevent use of the lumen 140 for its intended function. Such an elongate structure would then have to be disposed outside of both the cutting wire lumen 112 and the additional lumen 140. However, the limited amount of area of the elongate tubular member 104 outside of the cutting wire and additional lumens 112, 140 would force the elongate structure to have too small of a cross-sectional size such that the elongate structure would be too small and weak to support the elongate tubular member 104. As such, the elongate SMA structures of the present description may be better suited for multi-lumen sphincterotomes or other endoscopic medical devices that bend in a desired direction.

The elongate SMA structure 142 may encourage or facilitate the curling of the distal portion 108 in the desired direction, such as the 12 o'clock direction. To do so, the elongate SMA structure 142 may be formed so that its original, pre-deformed shape when unbiased is a longitudinally-curved shape. The term pre-deformed refers to when a bias is applied to the elongate SMA structure 142 to change the shape of the elongate SMA structure 142, and then when the bias is released, the elongate SMA structure 142 returns back to its original pre-deformed shape.

Regardless or independent of whether the handle assembly 122 is proximally pulling on the cutting wire 114, the elongate SMA structure 142 may be strong enough to curl the distal portion 108 of the elongate tubular member 104 and/or position the distal portion 108 in a curled position when the elongate SMA structure 142 is in its original, pre-deformed state. FIGS. 1A and 1B show the distal portion 108 in its curled position regardless of whether the cutting edge 120 is in its relaxed position (FIG. 1A) or taut position (FIG. 1B).

For some example configurations as shown in FIGS. 1A and 1B, the elongate SMA structure 142 may distally extend all the way to the distal end 146 of the body 110 of the elongate tubular member 104. In other example configurations, the elongate SMA structure 142 may not distally extend all of the way to the distal end 146, and instead, may distally extend past the anchor point 118 but terminate before the distal end 146, or alternatively may distally extend up to and terminate at the anchor point 118. In still other example configurations, the elongate SMA structure 142 may distally extend to a position before the anchor point 118, such as in between the opening 116 and the anchor point 118. Additionally, for the configuration shown in FIGS. 1A and 1B, the elongate SMA structure 142 may proximally extend past the opening 116. In other example configurations, however, the elongate SMA structure 142 may proximally extend up to and terminate at the opening 116, or may proximally terminate at a point distal the opening 116. In general, the elongate SMA structure 142 may longitudinally extend alongside at least a portion of the cutting edge 120.

Additionally, the elongate SMA structure 142 may be circumferentially oriented in the additional lumen 140 such that its radial direction of curvature is radially-aligned with the cutting edge 120. To illustrate, FIG. 3 shows an example cross-sectional profile of the sphincterotome 100 taken along line 1A-1A of FIG. 1A, where the distal end of the cutting edge 120 is affixed to the body 110 at the anchor point 118. The cutting edge 120 may radially extend away from the center of the elongate tubular member 104 in a direction denoted by arrow 302 in FIG. 3. The direction 302 in which the cutting edge 120 radially extends is also the desired 12 o'clock direction for cutting the sphincter muscle. Accordingly, in order to cut in the 12 o'clock direction, it is desirable for the distal portion 108 to curl in the direction denoted by arrow 302. As such, the elongate SMA member 142 may be circumferentially oriented in the additional lumen 140 to radially curve in the direction 302 so that the distal portion 108 curls in the desired direction 302.

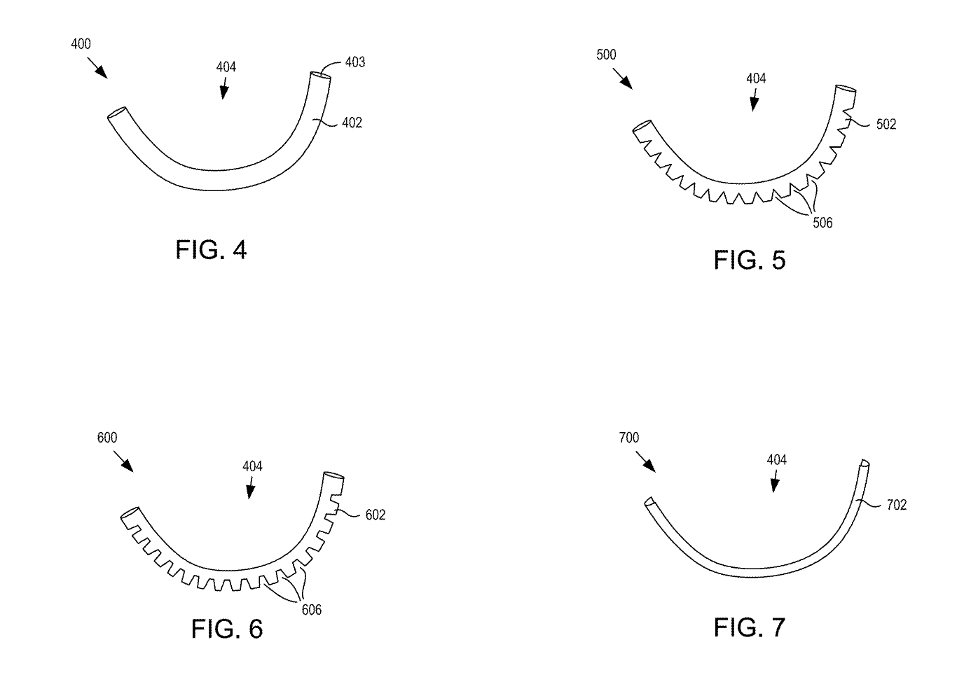

FIGS. 4-7 show example configurations of the elongate SMA structure 142. Each of the configurations shows the elongate SMA structure 142 in its longitudinally-curved pre-deformed shape. Referring to FIG. 4, in one example configuration, the elongate SMA structure 142 may be configured as an elongate tubular member 400 having a solid and contiguous body 402 over a longitudinal length of the elongate tubular member 400, and a lumen 403 longitudinally extending in the body 402. The curvature of the elongate tubular member 400 may define an inner area 404 in a plane in which the elongate tubular member 400 curves. For a given cross-sectional profile of the elongate tubular member 400, half of the outer surface of the body 402 may be considered to face toward the inner area 404, and the other half of the outer surface of the body 402 may be considered to face away from the inner area 404. In some example configurations, if the body 402 is too thick, the other half of the outer surface 402 may provide too much material for the tubular member 400 to curl the distal portion 108 as desired.

FIGS. 5 and 6 shows example configuration where the side facing away from the inner area 404 is weakened compared to the configuration of FIG. 4 by removing material in order to enhance the ability of the elongate SMA structure 142 to curl the distal portion 108. In particular, for each of the configurations shown in FIGS. 5 and 6, the elongate SMA structure 142 may be configured as an elongate tubular member 500 or 600 that has a series of notches 506 or 606 in a body 502 or 602. In addition, the notches 506 or 606 may be longitudinally aligned with each other. Also, for a given cross-sectional profile that includes a notch 506 or 606, a circumferential midpoint of the notch 506 or 606 may be 180-degrees from the midpoint of the half of the outer surface facing the inner area 404. Additionally, for some example configurations, a circumferential length of each of the notches 506 or 606 may be less than or equal to half the circumference of the tubular member 500, although longer circumferential lengths may be possible. For the configuration shown in FIG. 5, cuts made to form the notches 506 may be made relatively thin such that the notches 506, from a side perspective, are generally V-shaped when the elongate tubular member 500 is its pre-deformed curved shape. For the configuration shown in FIG. 6, cuts made to form the notches 606 may be comparatively thicker such that the notches 606, from a side perspective, are generally rectangular or trapezoidal.

FIG. 7 shows another example configuration in which an elongate structure 700 has a U-shaped, semi-circular shaped, or arc-shaped cross-sectional profile. As shown in FIG. 7, the elongate structure 700 may form its pre-deformed curve such that an inner surface as defined by the U-shape or arc-shape may face the inner area 404, and an outer surface as defined by the U-shape or arc-shape may face away from the inner area 404.

Other configurations or combination of configurations may be possible. For example, another example configuration may include some combination of the thinner notches 506 shown in FIG. 5 and thicker notches 606 shown in FIG. 6. Another example configuration may include notches in the body 702 of the elongate structure 700 having the U-shaped or arc-shaped cross-sectional profile.

As previously described, an example configuration for the elongate tubular member 104 may include two additional lumens, such as a wireguide lumen and a contrast lumen. Referring back to FIG. 3, the example cross-sectional profile of FIG. 3 shows a wireguide lumen 304 having a wireguide 306 movably disposed therein, and a contrast lumen 308. For the example configuration shown in FIG. 3, the wireguide lumen 304 may be the additional lumen that has disposed therein the elongate SMA structure 142. Also, the elongate SMA structure 142 shown in FIG. 3 has a circular cross-sectional profile, which may be representative of a cross-sectional profile of any of the elongate tubular member configurations 400, 500, and 600 shown in FIGS. 4, 5, and 6, respectively.

FIG. 8 shows another example cross-sectional profile of the sphincterotome 100 taken along line 1A-1A of FIG. 1A. The example cross-sectional profile shown in FIG. 8 is similar to that shown in FIG. 3, except that it shows the elongate SMA structure 142 as having a U-shaped, semi-circular, or arc-shaped cross-sectional profile, which may be representative of the cross-sectional profile of the elongate structure 700 of FIG. 7. For these configurations, ends of the U-shape, semi-circular, or arc-shape cross-sectional profile may form a line, denoted by dotted line 802, that is generally perpendicular to the direction in which the cutting edge 120 radially extends and the desired 12 o'clock curling direction.

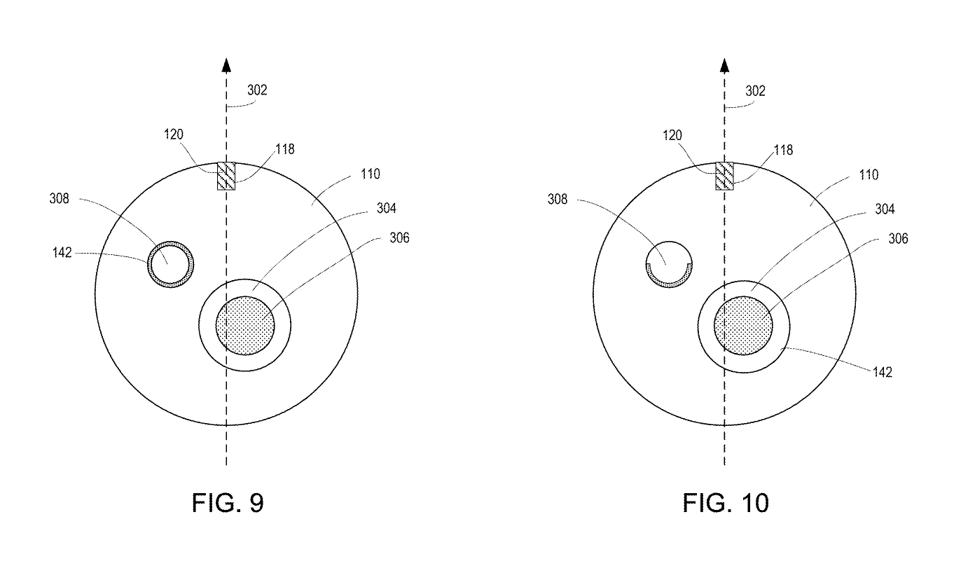

FIGS. 9 and 10 show other example cross-sectional profiles of the sphincterotome 100 taken along 1A-1A of FIG. 1A. The configuration shown in FIG. 9 is similar to that of FIG. 3, except that the elongate SMA structure 142 having the circular cross-sectional profile is disposed in the contrast lumen 308 instead of the wireguide lumen 304. Likewise, the configuration shown in FIG. 10 is similar to that of FIG. 8, except that the elongate SMA structure 142 having the U-shaped, semi-circular, or arc-shaped cross-sectional profile is disposed in the contrast lumen 308 instead of the wireguide lumen 304.

Referring back to FIGS. 1A and 1B, during operation, while the distal portion 108 is being delivered to a treatment site within a patient via a working channel of an endoscope, the distal portion 108 may be in a generally straightened configuration and the elongate SMA structure 142 may correspondingly be in a deformed straightened configuration due to the constraints of the working channel and/or due to the elongate tubular member 104 being disposed about a wireguide. If no wireguide is being used to deliver the distal portion 108 to the treatment site, then the distal portion 108 may begin to curl upon exiting the working channel of the endoscope at the treatment site due to the elongate SMA structure being able to return back to its original pre-deformed shape. Alternatively, if a wireguide is being used, then the distal portion 108 may begin to curl upon exiting the working channel and after the wireguide is proximally retracted away from the distal portion 108. The handle assembly 122 may be operated to pull the cutting edge 120 taut in order to cut the sphincter muscle, and may do so with the curling direction staying in the desired 12 o'clock direction due to the facilitation or encouragement of the curling provided by the elongate SMA structure 142.

The foregoing description of various embodiments of the invention has been presented for purposes of illustration and description. It is not intended to be exhaustive or to limit the invention to the precise embodiments disclosed. Numerous modifications or variations are possible in light of the above teachings. The embodiments discussed were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

1. A medical device comprising:
an elongate tubular member longitudinally extending from a proximal portion to a distal portion, the elongate tubular member comprising:
a body; and
a lumen longitudinally extending in the body from the proximal portion to the distal portion, wherein an inner surface of the body defines the lumen;
an elongate shape-memory alloy (SMA) structure that is disposed on the inner surface at the distal portion, the elongate SMA structure having a longitudinally-curved pre-deformed shape,
wherein the distal portion of the elongate tubular member is configured to be in a curled state due to the longitudinally-curved pre-deformed shape of the elongate SMA structure.

2. The medical device of claim 1, wherein the elongate SMA structure comprises a curved cross-sectional profile.

3. The medical device of claim 2, wherein a contour of the curved cross-sectional profile is defined by the inner surface of the body.

4. The medical device of claim 2, wherein the curved cross-sectional profile is an arc.

5. The medical device of claim 2, wherein the curved cross-sectional profile is a circle.

6. The medical device of claim 1, wherein the elongate SMA structure comprises an elongate tubular structure comprising a body and a lumen longitudinally extending in the body.

7. The medical device of claim 6, wherein the elongate tubular structure comprises a series of linearly-aligned notches.

8. The medical device of claim 7, wherein each of the linearly-aligned notches has a circumferential length less than or equal to half a circumferential length of the elongate tubular structure.

9. The medical device of claim 1, wherein the lumen comprises a first lumen, the medical device further comprising:
a second lumen longitudinally extending in the body; and
a cutting wire movably disposed and extending in the body.

10. The medical device of claim 9, wherein a cutting edge of the cutting wire longitudinally extends to outside of the body at the distal portion, and
wherein the elongate SMA structure longitudinally extends alongside the cutting edge.

11. The medical device of claim 1, wherein the elongate SMA structure comprises nitinol.

12. A medical device comprising:
an elongate tubular member longitudinally extending from a proximal portion to a distal portion, the elongate tubular member comprising:
a body;
a cutting wire lumen and an additional lumen, each longitudinally extending in the body from the proximal portion to the distal portion;
a cutting wire longitudinally extending in the cutting wire lumen, wherein a cutting edge of the cutting wire longitudinally extends outside of the body; and
an elongate shape-memory alloy (SMA) structure disposed on an inner surface of the body that defines the additional lumen, the elongate SMA structure having a longitudinally-curved pre-deformed shape,
wherein the distal portion of the elongate tubular member is configured to be in a curled position due to the longitudinally-curved pre-deformed shape of the elongate SMA structure.

13. The medical device of claim 12, wherein the elongate SMA structure comprises a curved cross-sectional profile.

14. The medical device of claim 13, wherein the curved cross-sectional profile is an arc.

15. The medical device of claim 13, wherein the curved cross-sectional profile is a circle.

16. The medical device of claim 12, wherein the elongate SMA structure comprises an elongate tubular structure comprising a body and a lumen longitudinally extending in the body.

17. The medical device of claim 16, wherein the elongate tubular structure comprises a series of linearly-aligned notches.

18. The medical device of claim 17, wherein each of the linearly-aligned notches has a circumferential length less than or equal to half a circumferential length of the elongate tubular structure.

19. The medical device of claim 12, wherein the additional lumen comprises a wireguide lumen.

20. The medical device of claim 12, wherein the additional lumen comprises a contrast lumen.

21. The medical device of claim 12, wherein the additional lumen comprises a first additional lumen, the elongate tubular member comprising a second additional lumen.
